(19) United States
(12) Patent Application Publication
(10) Pub. No.: US 2026/0320782 A1
(43) Pub. Date: Oct. 15, 2026

(54) APPARATUS AND METHOD FOR DETERMINING ARTERIAL INPUT FUNCTION OF DYNAMIC CONTRAST-BASED MAGNETIC RESONANCE IMAGING USING PHYSICS-INFORMED NEURAL NETWORK

(71) Applicant: KOREA ADVANCED INSTITUTE OF SCIENCE AND TECHNOLOGY, Daejeon (KR)

(72) Inventors: Sung-Hong PARK, Daejeon (KR); Muhammad Asaduddin, Daejeon (KR); Hyoseok Lee, Daejeon (KR)

(73) Assignee: KOREA ADVANCED INSTITUTE OF SCIENCE AND TECHNOLOGY, Daejeon (KR)

(21) Appl. No.: 19/463,524

(22) Filed: Jan. 29, 2026

(30) Foreign Application Priority Data
Feb. 25, 2025 (KR) 10-2025-0024207

(51) Int. Cl. G06T 7/00

(52) U.S. Cl. CPC G06T 7/0012; G06T 2207/10088; G06T 2207/20081; G06T 2207/20084; G06T 2207/30101

(57) ABSTRACT

The present disclosure provides an apparatus and method for determining an arterial input function of dynamic contrast-based magnetic resonance imaging using a physics-informed neural network. The method includes generating a plurality of tissue concentration functions based on a ground truth AIF, training the physics-informed neural network to output a common AIF based on the input tissue concentration functions, and determining a predicted AIF from in vivo data using the trained physics-informed neural network. Unlike existing AIF selection methods that have limitations in that pixels corresponding to arteries must be extracted from given data to determine AIF, a common AIF can be extracted from concentration functions of various tissues, so that AIF can be automatically and accurately determined even when selection of artery pixels is difficult or absent.

### BACKGROUND OF THE INVENTION

### Field of the Invention

The present disclosure relates to determination of an arterial input function of magnetic resonance imaging, and more particularly, to an apparatus and method for determining an arterial input function of dynamic contrast-based magnetic resonance imaging using a physics-informed neural network.

### Description of the Related Art

Perfusion information plays an important role in the diagnosis and treatment planning for various pathologies such as stroke, cerebral infarction, and epilepsy. In magnetic resonance imaging (MRI), perfusion information may be obtained through dynamic susceptibility contrast (DSC) MRI or dynamic contrast enhanced (DCE) MRI. To compute perfusion-related information from these dynamic contrast-based (DSC/DCE) MRI data, the radiologist needs to select an arterial input function (AIF) that represents the input to the tissue of interest. In addition, a mathematical operation such as deconvolution with AIF is applied to the concentration function derived from the tissue to calculate a tissue response function (TRF). TRF indicates whether blood flow is normal or abnormal.

Therefore, the selection of AIF is very important because it serves as a reference for all other tissue types within DSC/DCE MRI data.

AIF is an important process that determines the accuracy and reproducibility of perfusion maps, and various manual, semi-automatic, and automatic methods are being studied therefor. Manual methods require the radiologist to directly designate the AIF region, which may be time-consuming and tiring when multiple arteries supply blood to the tissues or when there is a large amount of data to analyze. Moreover, due to the low spatial resolution and time axis noise of DSC/DCE MRI, the selected region of interest (ROI) may be vulnerable to partial volume effects. Due to the limitations of manual methods, semi-automatic and automatic methods have been proposed, with clustering-based methods, gamma-variate function (GVF) fitting methods, criterion-based scoring methods, and deep learning methods as the main approaches.

However, these methods have been pointed out as having limitations such as clustering errors, pathological concentration changes, and incomplete data. In particular, deep learning-based approaches segment AIF regions or generate AIF and perfusion maps by utilizing dynamic MRI data, but have limitations due to dependence on training data.

In addition, existing AIF selection methods determine only one representative AIF (i.e., global AIF) within an organ of interest, such as the brain. Since arteries branch out and supply blood to various local regions, perfusion information may be matched much more accurately if the local AIF for each region may be accurately determined. However, due to technical limitations, accurate measurement of local AIF is currently considered practically impossible.

### SUMMARY OF THE INVENTION

The present disclosure has been made keeping in mind the problems encountered in the related art, and an object of the present disclosure is to provide an apparatus and method for determining an arterial input function of dynamic contrast-based magnetic resonance imaging using a physics-informed neural network.

An embodiment of the present disclosure provides a method of determining an arterial input function (AIF) of dynamic contrast-based magnetic resonance imaging (MRI) using a physics-informed neural network (PINN) capable of being performed by a computing apparatus. The method may include generating a plurality of tissue concentration functions based on a ground truth AIF, training the PINN to output a common AIF based on the input tissue concentration functions, and determining a predicted AIF from in vivo data using the trained PINN.

In addition, generating the tissue concentration functions may include determining the ground truth AIF and a plurality of residue functions depending on a physical model applied to the PINN, and determining the tissue concentration functions based on a convolution between the ground truth AIF and each of the residue functions.

In addition, the PINN may include a one-dimensional convolutional neural network (1D CNN) configured to receive the tissue concentration functions as input and to output the common AIF.

In addition, the 1D CNN may be configured to have a preset number of 1D convolutional layers.

In addition, each of the 1D convolutional layers may be configured to have a plurality of filters paired with a LeakyRELU layer and a 1D batch normalization layer.

In addition, determining the predicted AIF may include selecting an identical number of tissue concentration functions for input to the trained PINN from the in vivo data, and inferring the predicted AIF through the trained PINN based on the selected tissue concentration functions.

In addition, the tissue concentration functions selected from the in vivo data may be selected from a full range of the in vivo data or from a designated portion of the full range.

Another embodiment of the present disclosure provides an apparatus for determining an arterial input function (AIF) of dynamic contrast-based magnetic resonance imaging (MRI) using a physics-informed neural network (PINN). The apparatus may include a memory configured to store computer-executable instructions, and at least one processor connected to the memory and configured to execute the computer-executable instructions. In accordance with the computer-executable instructions, the processor may be configured to generate a plurality of tissue concentration functions based on a ground truth AIF, to train the PINN to output a common AIF based on the input tissue concentration functions, and to determine a predicted AIF from in vivo data using the trained PINN.

Still another embodiment of the present disclosure can provides a computer program stored on a computer-readable medium, including computer-executable instructions for performing the method of determining an arterial input function (AIF) of dynamic contrast-based magnetic resonance imaging (MRI) using a physics-informed neural network (PINN).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an exemplary block diagram illustrating a computing apparatus for determining an arterial input function of dynamic contrast-based magnetic resonance imaging using a physics-informed neural network according to an embodiment of the present disclosure;

FIG. 2 is an exemplary flowchart illustrating a process of determining an arterial input function of dynamic contrast-based magnetic resonance imaging using a physics-informed neural network according to an embodiment of the present disclosure;

FIG. 3 is an exemplary figure illustrating a physics-informed neural network for predicting an arterial input function (AIF) according to an embodiment of the present disclosure;

FIG. 4 is an exemplary figure illustrating the locations of randomly selected voxels during inference, with red dots indicating the selected locations;

FIG. 5 is an exemplary figure illustrating two representative arterial input functions (AIFs) derived from simulation data;

FIG. 6 is an exemplary figure illustrating representative AIF selection results from two patients; and

FIG. 7 is an exemplary figure illustrating perfusion maps and AIFs derived from patient data using the hand-picked method, the mean criterion-based method, and the proposed method.

### DETAILED DESCRIPTION OF THE INVENTION

Hereinafter, preferred embodiments of the present invention will be described in detail with reference to the attached drawings. First, when adding reference numerals to the components of each drawing, it should be noted that identical components are given the same numerals as much as possible even if they are shown in different drawings. In addition, when describing the present invention, if it is determined that a detailed description of a related known configuration or function may obscure the gist of the present invention, the detailed description is omitted.

Various aspects of the present invention are described below. It is to be understood that the inventions presented herein may be embodied in a wide variety of forms and that any specific structure, function, or both presented herein are merely exemplary. Based on the inventions presented herein, a person skilled in the art will understand that one aspect presented herein may be implemented independently of any other aspects, and that two or more of these aspects may be combined in various ways. For example, an apparatus may be implemented or a method may be practiced using any number of aspects described herein. In addition, such an apparatus may be implemented or such a method may be practiced using structures and/or functions other than, or in addition to, one or more aspects described herein.

The present disclosure relates to a simulation-based physics-informed neural network (PINN) approach. This approach may provide the ground truth arterial input function (AIF) and may train the network on a simulated dataset with a well-defined mathematical basis using a minimal number of approximate elements. Through this approach, the network may be trained to generate a plurality of tissue concentration functions based on a single well-defined AIF, and conversely, to output a common AIF by providing the diversely generated tissue concentration functions as input. The approach according to the present disclosure may provide a single AIF as output by inputting to the network, at the time of inference, a number of tissue response functions (TRFs), equal to the number of input TRFs used for training, selected from real in vivo data.

As will be described later, a model according to the present disclosure may be trained to predict AIF from a basic simulated dynamic contrast-based MRI time series and may be applied to patient data without fine-tuning or specific AIF pixel selection. In addition, as will be described later, the accuracy of the simulation-based approach according to the present disclosure may be confirmed to be superior to that of the criterion-based approach and the clustering-based approach.

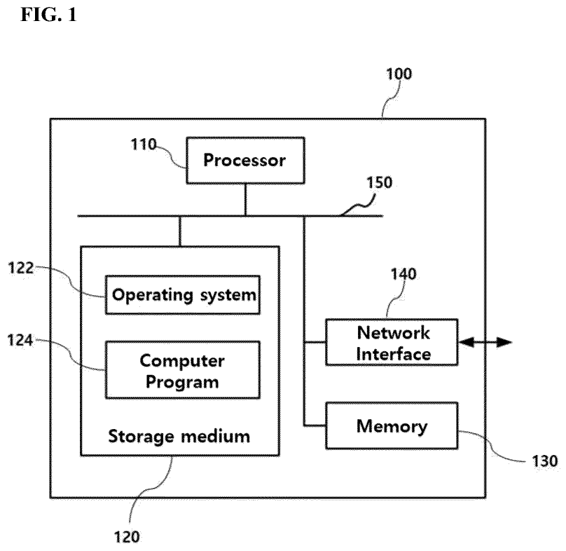

FIG. 1 is an exemplary block diagram illustrating a computing apparatus for determining an arterial input function of dynamic contrast-based magnetic resonance imaging using a physics-informed neural network according to an embodiment of the present disclosure.

As illustrated in FIG. 1, the computing apparatus 100 may include a processor 110, a storage medium 120, a memory 130, and a network interface 140, which may be connected to each other via a system bus 150.

An operating system (OS) 122 and a computer program 124 may be installed on the storage medium 120. The storage medium 120 may be a data storage device such as a hard disk or SSD (solid state drive) that may store computer programs and related data. The operating system 122 may be operating system software such as Windows, iOS, and Linux for operating the computing apparatus 100. The computer program 124 may include not only functional modules for determining an arterial input function of dynamic contrast-based magnetic resonance imaging using a physics-informed neural network according to the present disclosure, but also computer-executable instructions therefor. In addition, the computer program 124 may be loaded into the memory 130 so that it may be executed by the processor 110. The computer-executable instructions of the computer program 124, when executed by the processor 110, enable the processor 110 to perform a method of determining an arterial input function of dynamic contrast-based magnetic resonance imaging using a physics-informed neural network according to the present disclosure. The processor 110 may be configured to provide computing and control capabilities to support the execution of the entire computing apparatus 100. The processor 110 may be a data processor such as a CPU (central processing unit), an MPU (microprocessor unit), and an AP (application processor), and may be configured as a single processor or multiple processors. When configured as multiple processors, the processors 110 may operate as parallel processing processors. The network interface 140 may provide an interface that may be connected to an external device (e.g., a database storing magnetic resonance imaging-related data, a display device, another wired or wireless communication device that may be connected via a network, etc.) to communicate data.

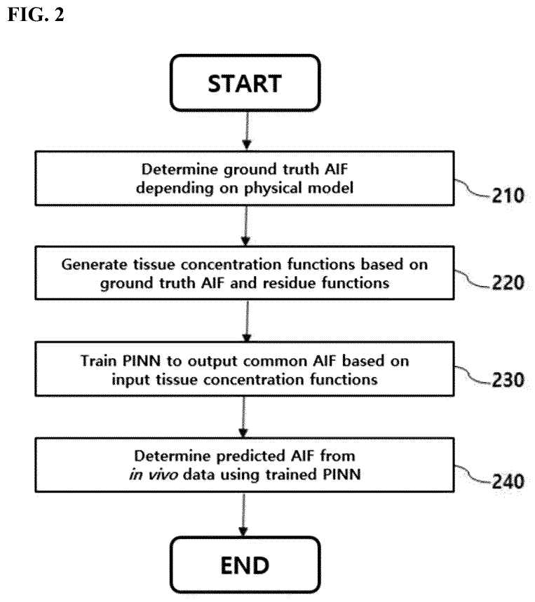

FIG. 2 is an exemplary flowchart illustrating a process of determining an arterial input function of dynamic contrast-based magnetic resonance imaging using a physics-informed neural network according to an embodiment of the present disclosure.

The computing apparatus 100 is able to train a physics-informed neural network (PINN) through the steps illustrated in FIG. 2 and to infer/output a predicted AIF from the input in vivo data using the trained PINN. To this end, determining the ground truth AIF depending on the physical model applied to the PINN (210) may be performed by the computing apparatus 100. The PINN may generate signals by pre-generating parameters corresponding to the ground truth followed by calculation through a physical model. Accordingly, the ground truth AIF and residue functions may be determined and input as simulation data for PINN training. Generating a plurality of tissue concentration functions based on the ground truth AIF and a plurality of residue functions (220) may be performed by the computing apparatus 100. Training the PINN to output a common AIF based on the input tissue concentration functions (230) may be performed by the computing apparatus 100. Determining a predicted AIF from in vivo data (e.g., in vivo measured magnetic resonance imaging (MRI) data) using the trained PINN (240) may be performed by the computing apparatus 100.

In addition, by the computing apparatus 100, a plurality of tissue concentration functions may be determined based on a mathematical operation of the ground truth AIF and residue functions determined depending on the physical model applied to the PINN. For example, such a mathematical operation may be a convolution operation between AIF and each of the residue functions, and when n residue functions are used, n tissue concentration functions may be calculated.

In addition, the PINN according to the present disclosure may include a one-dimensional convolutional neural network (1D CNN) configured to receive the generated tissue concentration functions as input and to output a single common AIF.

In addition, a 1D CNN may be configured to have a preset number of 1D convolutional layers. For example, when a 1D CNN has three 1D convolutional layers, each 1D convolutional layer may serve as a processing layer, resulting in three repeated processing layers.

In addition, each 1D convolutional layer may be configured to have a plurality of filters (e.g., 1000 filters) paired with a LeakyRELU layer and a 1D batch normalization layer.

In addition, by the computing apparatus 100, the same number of tissue concentration functions may be selected for input to the trained PINN from in vivo data. For example, when the PINN is trained using 1024 tissue concentration functions as input, the same number (i.e., 1024) of tissue concentration functions may be selected from in vivo data and input to infer AIF through the trained PINN. As such, 1024 pixels may be selected in the in vivo data space, and 1024 tissue concentration functions determined at each pixel may be provided as input to the trained PINN. By the computing apparatus 100, a predicted AIF may be inferred through the trained PINN based on the selected tissue concentration functions as described above.

In addition, by the computing apparatus 100, tissue concentration functions selected from in vivo data may be selected from the full range of in vivo data (e.g., the whole brain) in a predetermined selection manner (e.g., random selection, selection in a regular pattern, etc.) or from a designated portion of the full range (e.g., a specific brain region) in a predetermined selection manner (e.g., random selection, selection in a regular pattern, etc.). By the computing apparatus 100, a global AIF may be inferred by providing as input the tissue concentration functions selected from the full range of in vivo data, and also a local AIF may be inferred by providing as input the tissue concentration functions selected from a designated portion of the full range.

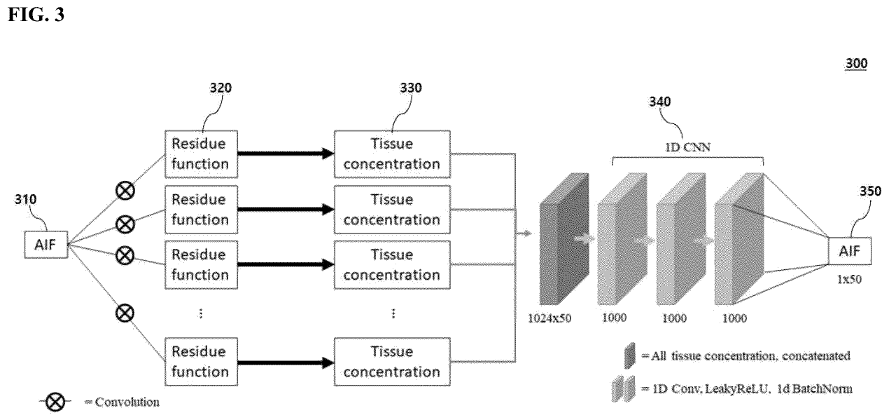

A more detailed description of a method of automatically determining an arterial input function (AIF) of dynamic contrast-based magnetic resonance imaging (MRI) according to the present disclosure, as described above, will be described below with reference to FIG. 3.

FIG. 3 is and exemplary figure illustrating a physics-informed neural network (PINN) for predicting an arterial input function (AIF) according to an embodiment of the present disclosure.

### 1. Simulation Data

To generate simulation data that provides an accurate ground truth AIF 310, the relationship between AIF and tissue concentration according to Equation 1 below may be used.

$${C}_{t} ( t ) = CBF * {R}_{t} ( t ) ⊗ {C}_{a} ( t ) \qquad [ Equation 1 ]$$

The AIF (Cₐ(t)) and tissue concentration function (Cₜ(t)) may be directly derived from real patient data using Equation 2 below, but the cerebral blood flow (CBF) and residue function (Rₜ) may be calculated by deconvolution between AIF and tissue concentration.

$$C ( t ) = - \frac{k}{TE} ln ( \frac{S ( t )}{{S}_{0}} ) \qquad [ Equation 2 ]$$

In this simulation, emphasis is placed on generating the AIF and residue functions (Rt) to generate a plurality of tissue concentration functions.

The model used to generate the AIF may be based on a gamma-variate function (GVF) with factors to reflect recirculation and dispersion according to Equations 3 to 5 below.

$${C}_{a , mono} ( t - t ) = 0.95 * {( \frac{t - t}{{t}^{′}} )}^{2.25} * {e}^{2.25 * ( 1 - \frac{t}{t} )} \qquad [ Equation 3 ]$$

$${C}_{a , bi} ( t ) = {C}_{a , mono} ( t - {t}_{arr} ) + 5 * {C}_{a , mono} ( t - ( {t}_{arr} + {t}_{recirc} ) ) ⊗ ( \frac{1}{{t}_{recirc}} {e}^{- \frac{t}{{t}_{recirc}}} ) \qquad [ Equation 4 ]$$

$${C}_{a , bi} ( t ) = 0 when t < {t}_{arr}$$

$${C}_{a} ( t ) = {C}_{a , bi} ( t ) ⊗ PSF ( 0 , ATT ) \qquad [ Equation 5 ]$$

These models may be parameterized by factors that specify the arrival time of the contrast bolus and the degree of dispersion experienced by the corresponding bolus before reaching the AIF region.

Equation 5 represents the AIF model used in these embodiments, which may be based on the dual-exponential model shown in Equation 4, and the dual-exponential model may be derived from the single-exponential model shown in Equation 3. Wherein, Tₐᵣᵣ is the arrival time of the first bolus and t_{recirc} is the arrival time of the recirculated bolus. In addition, ATT reflects the concentration transit time in the arteries. Various AIF forms may be generated by varying the parameters Tₐᵣᵣ and ATT.

The tissue response function (TRF) may be defined according to Equation 6 below. The TRF may be modeled by assuming that Rt reflects a single well-mixed compartment in the blood vessel, while being affected by dispersion as a contrast agent enters the tissue from the artery.

$$TRF = CBF * {R}_{t} ( t ) \qquad [ Equation 6 ]$$

A model for Rt may be modeled according to Equation 7 below.

$${R}_{t} ( t - {t}_{atd} ) = \frac{( dsp * {e}^{- dsp * ( t - {t}_{atd} )} - {e}^{\frac{t - {t}_{atd}}{MTT}} )}{\frac{dsp}{MTT} - 1} \qquad [ Equation 7 ]$$

Wherein, t_{atd} is the arterial transit delay time and dsp reflects the possible dispersion of the contrast bolus as it enters the tissue from the artery. Since the dispersion of the bolus should be proportional to the measured distance between the artery and tissue, the constant dsp is set to dsp=10/t_{atd}. Various Rₜ forms may be generated by varying CBF, t_{atd}, and MTT.

Using the model for the AIF 310 and the residue function 320 defined as described above, tissue concentration functions 330 may be calculated using Equation 1. To simulate whole brain dynamic contrast-based (DSC/DCE) MRI data, a plurality of residue functions may be convolved with a single AIF to generate a plurality of tissue concentration functions (FIG. 3). In this embodiment, 1024 unique tissue concentration functions are generated for each single AIF.

### 2. 1D Convolutional Neural Network (1D CNN)

FIG. 3 illustrates a deep learning network according to the present disclosure for predicting AIF. Taking into consideration the input and output forms, a one-dimensional convolutional neural network (1D CNN) 340 may be applied to the network to process simulation data. 1024 unique tissue concentration functions may be considered as a single input and processed to provide a single output. In the 1D CNN architecture of this embodiment, the input may be composed of 1024 channels of time-series data with a length of 50. The 1D convolutional layer of a 1D CNN may be configured to have 1000 filters paired with a LeakyRELU layer and a 1D batch normalization layer, and may be used as a single processing layer. This processing layer may be repeated three times, resulting in an output of a 1-channel time-series with a length of 50 representing the predicted AIF 350.

Training of 1D CNN may be performed exclusively using simulation data, and the AIF may be generated using tₐᵣᵣ ranging from 1 to 30 seconds and ATT ranging from 0 to 3 seconds. Each residue function Rₜ may be generated using CBF values ranging from 0 to 600 ml/100 g/min, MTT values ranging from 1.1 to 15 seconds, and t_{atd} values ranging from 1 to 7 seconds. During training, 1024 DSC MRI tissue time-series were used as input to the 1D CNN network, and one AIF time-series was generated as output. The error between the simulation-based AIF used to generate the input and the generated AIF was calculated using the mean absolute error (L1loss). This training continued until the training loss reached a plateau, at which point 1.5M AIF samples had been generated. In addition, the ADAM optimizer was used with a learning rate of 10⁻² for unnormalized data.

### 3. Patient Data

This study was approved by the Institutional Review Board (IRB), and DSC-MRI was performed on 20 patients (age range: 17-79 years; 10 females) diagnosed with ischemic stroke. Imaging was performed using a 3T MRI scanner equipped with a head coil. The contrast agent was used at a concentration of 1 mmol/ml with an average injection volume of 4.5 ml. A 2D gradient-recall-echo echo-planar imaging (EPI) sequence was used for DSC-MRI acquisition, with the following acquisition parameters: repetition time/echo time (TR/TE)=1751/30 ms, flip angle=90°, matrix size=128×128, number of slices=24, field of view (FOV)=230×230 mm², slice thickness=4 mm, scan direction=axial, number of dynamic scans=50. The number of slices initially acquired was 24, but was reduced to 20 to retain only slices containing anatomical structures, and slices containing only background noise were excluded.

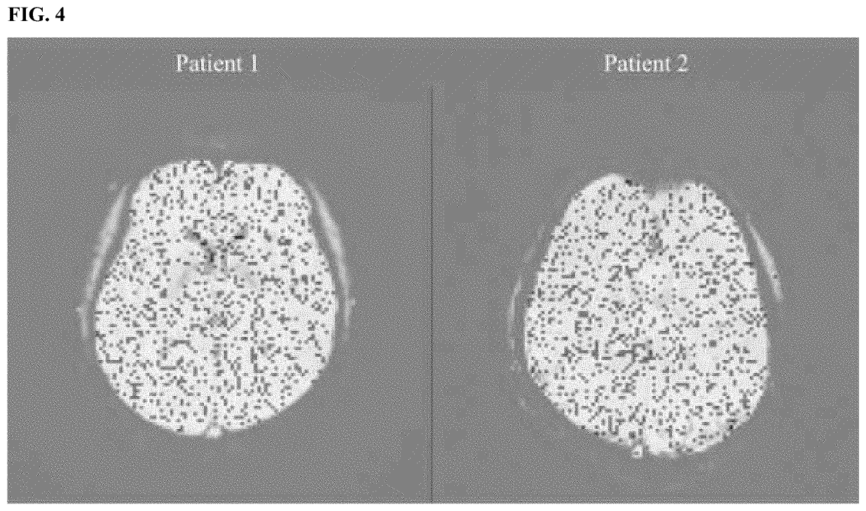

During the inference process of patient data, 1024 time series were extracted to be applied to the 1D CNN. To this end, a mask based on intensity thresholds was applied to remove the background. After applying the mask, 1024 voxels were randomly selected, most of which were located inside the brain (FIG. 4). In FIG. 4, red dots indicate selected locations. 1024 time series were extracted from these voxels and used as input to the CNN model.

### 4. Evaluation

The AIF prediction method using simulation-based PINN according to the present disclosure was compared with criterion-based and clustering-based automatic AIF selection algorithms using a scoring system according to Equation 8 below.

$$Score = peak + ( 50 - FWHM ) + ( 50 - {t}_{peak} ) \qquad [ Equation 8 ]$$

In the criterion-based algorithm, each time series may be ranked and the highest-scoring time series may be selected as the AIF. Additionally, an alternative AIF may be created by averaging the top five highest-scoring time series. These AIFs are referred to herein as “criterion-based” AIFs and “mean criterion-based” AIFs.

In the clustering-based method, all tissue concentration functions are initially divided into five clusters based on their peak times and AUC (area under curve). The cluster with the combination of earliest peak time and highest AUC is further divided into five clusters using the same features. The resulting clusters with the earliest peak time and highest AUC are averaged, and this averaged concentration function may be used as the AIF. In both the initial and second clustering steps, the K-means clustering algorithm was used.

Additionally, for patient data, AIF was also segmented from specific anatomical regions. In many guidelines, the M1 or M2 section of the middle cerebral artery (MCA) territory is considered a good area for AIF segmentation. The AIF measured in this manner is referred to herein as “hand-picked” AIF.

The accuracy of each method was tested on simulation data where the ground truth AIF was known. As such, hand-picked AIF is not available because the simulation data does not include any specific arterial region. To measure the accuracy of each AIF selection method, the normalized root mean square error (NRMSE) and peak lag were calculated. Peak lag may be defined as the difference between the peak positions of the ground truth AIF and the automatically derived AIF. In addition, each derived AIF was additionally used to calculate the perfusion parameters CBF, Tmax, and MTT of each tissue in the simulation data.

True CBF, Tmax and MTT values are known based on simulations, enabling the accuracy of each AIF selection method to be measured. NRMSE was calculated between true and estimated CBF, Tmax, and MTT.

Meanwhile, the performance of each method was also tested using patient data, to which the proposed method of the present disclosure, the criterion-based method, the mean criterion-based method, the clustering method, and the hand-picked method may be applied. Although none of these methods may be considered absolute ground truth, the hand-picked method was used as a reference AIF because the selected AIF has an evident physical relationship with the MCA region. These automatic methods were compared with the reference AIF with regard to correlation and resulting perfusion maps. It was assumed that hand-picked/reference AIFs may suffer from partial volume effects, but some properties of the absolute true AIF should still be reflected. Specifically, the true AIF should have a peak time that is within the enhanced period of the hand-picked AIF, but slightly earlier due to the partial volume effect of the hand-picked AIF. Thus, AIF derived from automatic methods should also show peak concentrations at earlier points within this enhanced period. In the comparison of perfusion maps, the AIF derived from each method, including the reference AIF, was used to process DSC MRI data into perfusion maps of CBF, MTT, and Tmax. The conventional oscillation index singular value decomposition (oSVD) method and the recently proposed PINN-based deconvolution method (SPINNED) were used to compute the perfusion maps.

### 5. Evaluation Results

### (1) Evaluation of Simulation Data

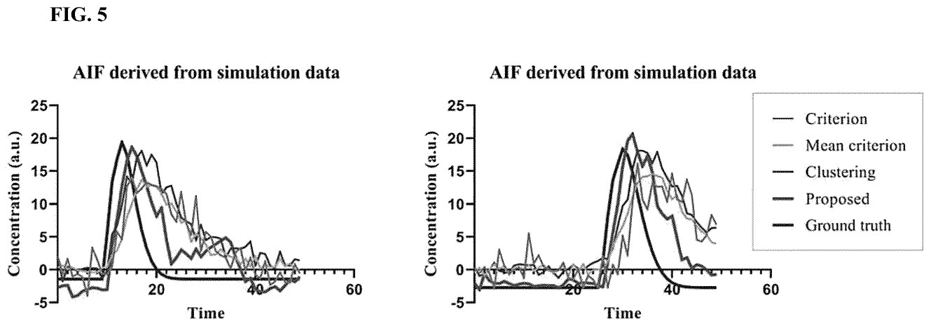

The accuracy of automatic AIF selection methods, including the proposed method of the present disclosure, was quantitatively evaluated using simulation data where the true AIF is known. Two representative examples of AIFs derived from the ground truth, criterion-based, mean criterion-based, clustering and proposed methods of the present disclosure are shown in FIG. 5. The AIF derived from the proposed method of the present disclosure (red line in FIG. 5) showed the closest peak concentration and peak time to the ground truth (blue line in FIG. 5) among all tested automatic AIF selection methods.

Quantitatively, the proposed method of the present disclosure showed the best accuracy in terms of NRMSE (0.1363±0.0409) and peak lag (3.5680±1.6996) among all tested methods, consistent with visual inspection (Table 1). As is apparent from Table 1 below, the proposed method of the present disclosure showed the lowest NRMSE and the shortest peak lag, indicating that it is the most accurate among the tested methods.

TABLE 1
QUANTITATIVE COMPARISON OF THE ACCURACY OF AUTOMATIC
AIF SELECTION METHODE AGAINST SIMULATION DATA

| Methods | NRMSE | Peak lag |
|---|---|---|
| Criterion-based | 0.1724 ± 0.0502 | 5.0880 ± 4.0065 |
| Mean criterion | 0.1816 ± 0.0509 | 9.0720 ± 3.4192 |
| Clustering | 0.2146 ± 0.0695 | 16.2880 ± 12.1073 |
| Proposed | 0.1363 ± 0.0409 | 3.5680 ± 1.6996 |

Perfusion parameters for each of the simulated tissues were also calculated using each of the automatically derived AIFs. The proposed method of the present disclosure showed the best accuracy for all perfusion parameters, including the best NRMSE equal to 0.0036±0.0014 for MTT (Table 2).

TABLE 2
QUANTITATIVE COMPARISON OF PERFUSION
PARAMETERS FOR SIMULATION DATA

| | | Mean criterion | Clustering | |
|---|---|---|---|---|
| | Criterion-based | NRMSE | | Proposed |
| CBF | 0.0547 ± 0.0205 | 0.0699 ± 0.0244 | 0.0616 ± 0.0273 | 0.0474 ± 0.0198 |
| Tmax | 0.0059 ± 0.0020 | 0.0069 ± 0.0018 | 0.0077 ± 0.0032 | 0.0048 ± 0.0019 |
| MTT | 0.0041 ± 0.0016 | 0.0048 ± 0.0018 | 0.0047 ± 0.0018 | 0.0036 ± 0.0014 |

### (2) Evaluation of Patient Data

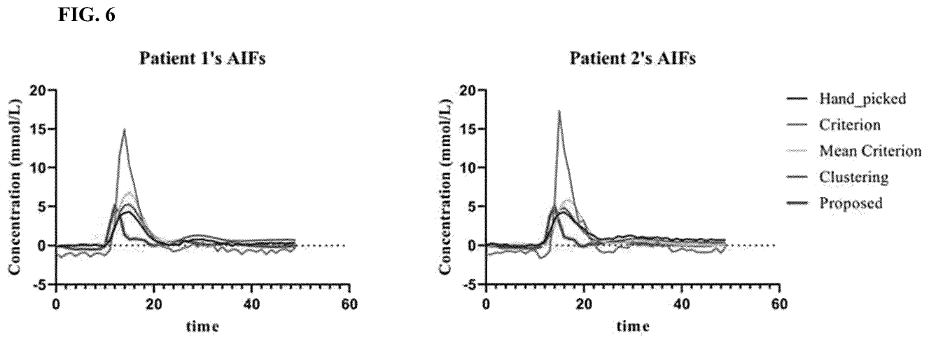

FIG. 6 shows representative AIF selection results from two patients using the hand-picked, criterion-based, mean criterion-based, clustering and proposed methods. Typically, the AIF of the proposed method reached the peak time earliest, followed by the reference (hand-picked) AIF. The peak times of other methods were generally later than those of the two methods mentioned above. The FWHM of the AIF of the proposed method was generally the narrowest. Although the peak height of the AIF of the proposed method was higher than that of the reference method, the criterion-based and mean criterion-based AIFs generally showed higher peaks than the two methods mentioned above. These visual observations were consistent with quantitative analyses for all tested subjects (Table 3).

TABLE 3
QUANTITATIVE COMPARISON OF SCORE
ELEMENTS FOR PATIENT DATA

| Methods | Peak Time | FWHM | Peak Height |
|---|---|---|---|
| Hand-picked | 18.45 ± 4.03 | 6.41 ± 174 | 4.07 ± 0.96 |
| Criterion-based | 17.00 ± 5.15 | 3.21 ± 136 | 14.30 ± 4.73 |
| Mean criterion- | 18.50 ± 5.51 | 4.90 ± 1.32 | 5.40 ± 1.72 |
| based | | | |
| Clustering | 21.20 ± 8.16 | 11.25 ± 12.14 | 4.92 ± 1.29 |
| Proposed method | 16.35 ± 3.88 | 2.87 ± 0.59 | 4.49 ± 0.81 |

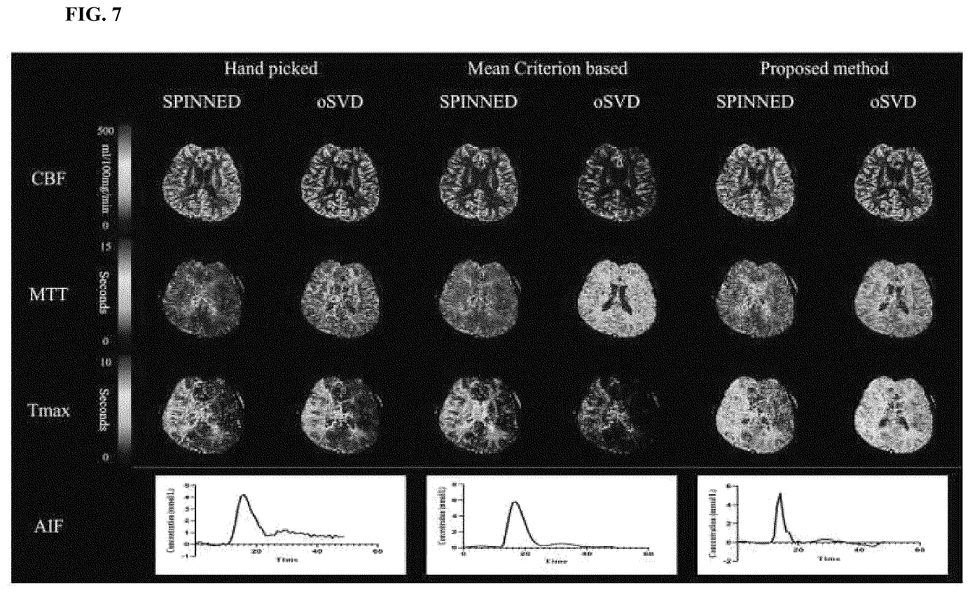

Perfusion maps derived using each of the AIFs selected from the patient data are shown in FIG. 7. Perfusion maps derived from the proposed method were able to highlight perfusion anomalies (Tmax>6 seconds) that were scarcely observed when using AIFs obtained by the hand-picked or mean criterion-based method. This was true regardless of how the perfusion map was calculated. The CBF values derived using AIFs from the proposed method, the criterion-based method, and the mean criterion-based method showed good agreement with the CBF values derived using hand-picked AIFs (Table 4). Table 4 below shows the correlation coefficients of CBF values derived using hand-picked AIFs and CBF values derived using automatically selected AIFs.

TABLE 4
CORRELATION COEFFICIENTS OF CBF VALUES

| Method | Correlation coefficient |
|---|---|
| Criterion-based | 0.9273 ± 0.0534 |
| Mean criterion-based | 0.9801 ± 0.0258 |
| Clustering | 0.9824 ± 0.0301 |
| Proposed method | 0.9855 ± 0.0129 |

Among the automatic selection methods, the proposed method showed the highest correlation with the hand-picked method (Table 4), suggesting a strong relationship between the AIF derived from the proposed method and the AIF derived from the MCA region, even though there is no direct connection between the two.

As described above, the present disclosure relates to a novel method of predicting AIF from dynamic contrast-based MRI data using a simulation-based physics-informed neural network (PINN). Simulation data was used to generate AIFs based on GVF and to generate various tissue time series derived from the generated AIFs. Modeling using GVF offers the advantage of fitting individual data more accurately by using fewer parameters. Additionally, both exponential functions and GVFs offer high computational efficiency, making them suitable for generating large-scale simulation data. This approach enabled the generation of large-scale simulation data for training. Although clinical application of existing deep learning methods is difficult due to large data requirements, this method utilizes GVF to generate large-scale simulation data for training, which enables the development of more accurate deep learning models.

A single AIF used to generate the tissue time series was successfully predicted using the proposed 1D CNN architecture, which showed greater accuracy than previously reported automatic AIF selection approaches in the aforementioned tests. As is apparent from Table 1, compared to other tested methods, simulation data showed that the proposed method achieved the lowest NRMSE and standard deviation with regard to the ground truth. As is apparent from Table 2, when deriving perfusion parameters, the proposed method showed the lowest NRMSE and standard deviation, and verified high accuracy and reliability compared to previously reported automatic AIF selection methods. Moreover, since training data already reflected the form and amplitude of AIF, the proposed method does not require additional post-processing steps such as rescaling, enabling more accurate predictions.

The proposed method for AIF selection was also shown to be applicable to patient data, and automatic AIF selection methods were compared with hand-picked AIFs obtained from the MCA region. Table 3 shows comparison of the criteria used to evaluate AIF, specifically peak time, FWHM, and peak height. As shown in Table 3, the proposed method has the shortest peak time and the narrowest FWHM. However, the criterion-based method results in higher peak heights because it prioritizes the selection of the highest values. Given that the criterion-based peak height is approximately three times greater than the reference peak height and that the CBF falls in the range of 0 to 500, these results are outside the expected range. Therefore, it may be concluded that the amplitude is predicted more appropriately by the proposed method. Perfusion maps derived from the proposed AIF selection method showed good agreement with perfusion maps derived from the hand-picked method and could even highlight perfusion anomalies (Tmax>6 seconds) that were difficult to observe when using other methods. The strong correlation between the perfusion maps from the reference hand-picked AIF and the perfusion maps from the proposed method's AIF suggests that the AIFs derived using the proposed method have a strong relationship with concentrations in the MCA region, even though there is no direct connection between the two.

The AIF may be inferred from many tissue concentration functions by the proposed method. The idea is to predict a single concentration function that may be used to regenerate all other tissue concentration functions using appropriate tissue response functions. This assumption is consistent with the current conventional approach, where a single AIF is used to describe the concentration functions of all tissues in the imaging field. This is referred to as a global AIF. The use of the global AIF may result in partial volume effects (PVE) due to the limited resolution of DSC-MRI. In addition, the distance between the AIF measurement site and the tissue of interest may result in bolus delay and dispersion, which in turn introduces errors in CBF measurements.

Various strategies have been reported to correct PVE. According to a paper by van Osch et al., the signals measured during the bolus passage traced an inward spiral trajectory in the complex plane. When the signal consists of pure arterial blood, the center of the spiral trajectory is located at the origin of the complex plane. However, when partial volume effects are present, contributions from outside the blood vessel shift the center away from the origin. Taking advantage of these characteristics, the paper by Bleeker et al. proposed a method of correcting PVE when measuring AIF from the internal carotid artery.

However, this method is only applicable when the blood vessels are parallel to the main magnetic field of the MRI, and since this method is phase-based, it cannot be applied when measuring AIF using amplitude-based techniques.

Hence, additional criteria were proposed to avoid selection bias caused by PVE. These criteria are based on tracer kinetic principles and are used to calculate cerebral blood volume (CBV) across the whole brain, suggesting that the ratio of the post-bolus steady-state concentration to the area under the curve (SS:AUC) should remain constant.

However, these methods are limited by the requirement for data that are insensitive to the Ti effect. Although these methods are limited by certain conditions, the proposed method may provide the advantage of extracting an optimal AIF by considering the overall characteristics of the brain regardless of resolution.

To handle bolus delay and dispersion, it is advantageous to use a local AIF. Although previous studies have proposed automatic local AIF methods, the accuracy of local AIFs determined based on criteria from local pixel intensities may be limited due to a much smaller number of pixels. The proposed method according to the present disclosure may be extended to estimate AIFs in local regions and is applicable to tissues within these regions.

The proposed method only requires a set of tissue concentration functions as input and does not require additional segmentation, thereby achieving equally fast processing times for both global and local AIFs. Local AIFs may be desirable when there is significant dispersion or delay between the reference arterial territory (primarily the MCA) and the tissue of interest. In these cases, the AIF estimated from MCA is no longer related to the concentration in the tissue of interest.

The proposed method leverages the mathematically modeled properties of GVF to enable significantly accurate and efficient data analysis compared to existing AIF evaluation techniques. GVF is a mathematically sophisticated model that minimizes reliance on large-scale clinical data and also provides stable computational performance. Therefore, GVF may achieve high accuracy with very low computational power and is applicable even in resource-limited environments. Training may be completed in less than 1 hour using typical research equipment such as an NVIDIA GeForce GTX 1080Ti GPU, an AMD Ryzen 7 7700X 8-Core Processor (4.50 GHz), and 16 GB DDR5 RAM. This efficiency not only reduces training time, but also lowers research costs and provides broad accessibility to a wide range of researchers. Additionally, ease of fine-tuning allows for versatile applications in a variety of scenarios, further improving accessibility. Recent advances in deep learning (with PINNs) also provide new and more accurate methods for performing deconvolution operations and generating accurate perfusion maps. By combining PINNs for both AIF selection and deconvolution, robust DSC perfusion maps may be obtained without the need for radiologist input or training with real data. In this context, the proposed method may offer significant advantages over existing techniques by maintaining high efficiency and reducing resource consumption, and may be more applicable in practical settings.

In summary, the present disclosure relates to a novel method of generating optimal AIF from dynamic contrast-based MRI data using a simulation-based physics-informed neural network (PINN). These models may be trained to predict AIF from baseline simulated dynamic contrast-based MRI time series and applied to patient data without fine-tuning or specific AIF pixel selection. The proposed method is capable of generating AIF much faster than existing extraction methods and has demonstrated higher accuracy in in silico experiments. When applied to real patient data, the proposed method yielded AIFs that exhibited earlier and higher peaks (closer to the expected peak characteristics) while remaining within the same enhanced period as the hand-picked AIFs. Additionally, perfusion maps from the proposed method showed higher correlation with perfusion maps from the reference hand-picked AIFs than other automatic AIF selection methods.

Moreover, the proposed method enables easy fine-tuning and wide applicability because it does not rely on clinical data for training, and the shortened inference time enables real-time applications. The proposed method may also be applied to generate brain region-specific local AIFs.

Based on the accuracy, stability, wide applicability, and scalability, the proposed method has the potential to become a standard for practical use.

As is apparent from the foregoing, according to the present disclosure, unlike existing AIF selection methods that have limitations in that pixels corresponding to arteries must be extracted from given data to determine AIF, a common AIF can be extracted from the concentration functions of various tissues, thereby enabling automatic and accurate determination of AIF even when selection of artery pixels is difficult or absent.

In addition, according to the present disclosure, a method of very accurately measuring local AIF, which was conventionally considered difficult to solve due to technical difficulties, can be provided.

It is understood that any particular order or hierarchy of steps in any presented processes is merely an embodiment of exemplary approaches. It is to be understood that the specific order or hierarchy of steps in the processes within the scope of the present invention may be rearranged based on design priorities. The attached method claims provide elements of various steps in an exemplary order, but are not meant to be limited to the specific order or hierarchy presented.

The terms “component,” “unit (or part),” “module,” “system,” and the like, as used herein, may refer to a computer-related entity, hardware, firmware, software, a combination of software and hardware, or an execution of software. For example, a component may be, but is not limited to, a process running on a processor, a processor, an object, a thread of execution, a program, and/or a computer. For example, both an application running on a computing apparatus and the computing apparatus itself may be regarded as components. One or more components may reside within a processor and/or thread of execution, and a component may be localized within one computer or distributed between two or more computers. Additionally, these components may be executed from various computer-readable media having various data structures stored therein.

The description of the presented embodiments is provided to enable any person skilled in the art to use or practice the present invention. Various modifications to these embodiments will be apparent to those skilled in the art, and the general principles defined herein may be applied to other embodiments without departing from the scope of the present invention. Accordingly, the present invention is not limited to the embodiments presented herein, but should be construed in the broadest scope consistent with the principles and novel features presented herein.

### DESCRIPTION OF REFERENCE NUMERALS

100: Computing apparatus
110: Processor
120: Storage medium
122: Operating system
124: Computer program
130: Memory
140: Network interface
150: System bus
300: Physics-informed neural network
310: Ground truth AIF
320: Residue function
330: Tissue concentration function
340: 1D CNN
350: Predicted AIF

What is claimed is:

1. A method of determining an arterial input function (AIF) of dynamic contrast-based magnetic resonance imaging (MRI) using a physics-informed neural network (PINN) capable of being performed by a computing apparatus, comprising:
generating a plurality of tissue concentration functions based on a ground truth AIF;
training the PINN to output a common AIF based on the input tissue concentration functions; and
determining a predicted AIF from in vivo data using the trained PINN.

2. The method according to claim 1, wherein generating the tissue concentration functions comprises:
determining the ground truth AIF and a plurality of residue functions depending on a physical model applied to the PINN; and
determining the tissue concentration functions based on a convolution between the ground truth AIF and each of the residue functions.

3. The method according to claim 1, wherein the PINN comprises a one-dimensional convolutional neural network (1D CNN) configured to receive the tissue concentration functions as input and to output the common AIF.

4. The method according to claim 3, wherein the 1D CNN is configured to have a preset number of 1D convolutional layers.

5. The method according to claim 4, wherein each of the 1D convolutional layers is configured to have a plurality of filters paired with a LeakyRELU layer and a 1D batch normalization layer.

6. The method according to claim 1, wherein determining the predicted AIF comprises:
selecting an identical number of tissue concentration functions for input to the trained PINN from the in vivo data; and
inferring the predicted AIF through the trained PINN based on the selected tissue concentration functions.

7. The method according to claim 6, wherein the tissue concentration functions selected from the in vivo data are selected from a full range of the in vivo data or from a designated portion of the full range.

8. An apparatus for determining an arterial input function (AIF) of dynamic contrast-based magnetic resonance imaging (MRI) using a physics-informed neural network (PINN), comprising:
a memory configured to store computer-executable instructions; and
at least one processor connected to the memory and configured to execute the computer-executable instructions,
wherein, in accordance with the computer-executable instructions, the processor is configured to generate a plurality of tissue concentration functions based on a ground truth AIF, to train the PINN to output a common AIF based on the input tissue concentration functions, and to determine a predicted AIF from in vivo data using the trained PINN.

9. The apparatus according to claim 8, wherein the processor is further configured to determine the ground truth AIF and a plurality of residue functions depending on a physical model applied to the PINN, and to determine the tissue concentration functions based on a convolution between the ground truth AIF and each of the residue functions.

10. The apparatus according to claim 8, wherein the PINN comprises a one-dimensional convolutional neural network (1D CNN) configured to receive the tissue concentration functions as input and to output the common AIF.

11. The apparatus according to claim 10, wherein the 1D CNN is configured to have a preset number of 1D convolutional layers.

12. The apparatus according to claim 11, wherein each of the 1D convolutional layers is configured to have a plurality of filters paired with a LeakyRELU layer and a 1D batch normalization layer.

13. The apparatus according to claim 8, wherein the processor is further configured to select an identical number of tissue concentration functions for input to the trained PINN from the in vivo data, and to infer the predicted AIF through the trained PINN based on the selected tissue concentration functions.

14. The apparatus according to claim 13, wherein the tissue concentration functions selected from the in vivo data are selected from a full range of the in vivo data or from a designated portion of the full range.

15. A computer program stored on a computer-readable medium, comprising computer-executable instructions for performing the method according to claim 1.
